(19) United States
(12) Patent Application Publication
(10) Pub. No.: US 2008/0200781 A1
(43) Pub. Date: Aug. 21, 2008

(54) Glucose Sensor

(75) Inventors: Maarten Marinus Johannes Wilhelmus Van Herpen, Eindhoven (NL); Marcello Leonardo Mario Balistreri, Eindhoven (NL); Cristian Presura, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/914,976

(22) PCT Filed: May 19, 2006
(86) PCT No.: PCT/IB2006/051599

§ 371 Date: Nov. 20, 2007

(30) Foreign Application Priority Data
May 24, 2005 (EP) 05300403.2

(51) Int. Cl. A61B 5/00

(52) U.S. Cl. 600/316

(57) ABSTRACT

A system for the non-invasive measurement of glucose concentration in a live subject is disclosed. The system exploits the metabolic heat conformation method, and comprises temperature sensing means for measuring the body heat in respect of the subject and means for measuring the concentration of haemoglobin and oxygenated haemoglobin in the blood of the subject. The system further comprises irradiating means for irradiating a portion of the live subject, a detector for collecting the measuring beam reflected by the live subject, means for determining from the reflected measuring beam, the blood flow velocity in respect of the live subject, and means for determining glucose concentration in the live subject as a function of the body heat, the haemoglobin and oxygenated haemoglobin concentrations and the blood flow velocity.

The present invention relates to the non-invasive measurement of glucose concentration in a live subject and, more particularly, to the non-invasive measurement of blood glucose concentration using the so-called metabolic heat conformation method.

The non-invasive determination of blood glucose concentration using the known Metabolic Heat Conformation (MHC) method relies on the measurement of the oxidative metabolism of glucose, from which the blood glucose concentration can be inferred. Body heat generated by glucose oxidation is based on the subtle balance of capillary glucose and oxygen supply to the cells of a tissue. The MHC method exploits this relationship to estimate blood glucose by measuring the body heat and the oxygen supply. The relationship can be represented in an equation as:

[Glucose concentration]=Function[Heat generated, Blood flow rate, Hb, HbO₂]

where Hb and HbO₂ represent the haemoglobin and oxygenated haemoglobin concentrations, respectively.

The heat generated (i.e. body heat) is measured with a thermometer and the Hb and HbO₂ concentrations are typically determined from the spectral reflectivity of the skin. Using the known MHC method, the blood flow rate is estimated from the thermal conductivity of the skin, and this thermal conductivity is detected by measuring the heat transferred through the skin from the tissue sample, such as a fingertip, to two thermistors.

Cho et al. (“Non-invasive Measurement of Glucose by Metabolic Heat Conformation Method”, Clinical Chemistry, 50(10), pp 1894-1898, (2004)) have demonstrated that the MHC method can indeed be used for non-invasive glucose detection. The blood flow rate is calculated by determining the heat conductivity and convection. However, measurement of thermal conductivity depends on the water content of the tissue sample. Unless the water content is determined first, the error associated with the calculated blood flow rate can become quite large.

The water concentration of the tissue sample can be measured by looking at the variation of the thermal conductivity during the first 2 seconds of contact. However, the problem of determining blood flow rate then becomes one of accurately determining more than one parameter. Thus, it is an object of the present invention to provide a system for the non-invasive measurement of glucose concentration in a live subject, wherein the determination of blood flow rate is performed directly, and is dependent on a single parameter to improve the accuracy in the measurement of glucose concentration using the MHC method.

In accordance with the present invention there is provided a system for non-invasive measurement of glucose concentration in a live subject, said system comprising temperature sensing means for measuring body heat in respect of said subject, means for measuring the concentration of haemoglobin and oxygenated haemoglobin in the blood of said live subject, irradiating means for generating a measuring beam and irradiating therewith a portion of said live subject, detector means for collecting measuring beam radiation reflected by said live subject, means for determining from said reflected measuring beam radiation blood flow velocity in respect of said live subject, and means for determining glucose concentration in said live subject as a function of said body heat, said haemoglobin and oxygenated haemoglobin concentrations, and said blood flow velocity.

In a first exemplary embodiment of the present invention, said means for determining blood flow velocity comprises self-mixing interferometry measurement means wherein said blood flow velocity is determined according to interference of radiation incident on said portion of said live subject with radiation reflected therefrom.

In one preferred embodiment of the present invention, using self-mixing interferometry measurement means, means may be provided for determining the rate of oscillation of a signal derived from the measuring beam radiation collected by said detector means, said rate of oscillation being dependent on changes in the speckle pattern derived from said measuring beam radiation collected by said detector means, and being representative of the heartbeat of said live subject. Thus, beneficially, the heartbeat and blood velocity are measured substantially simultaneously. In this case, the glucose measurement may be more accurate because real time-varying blood flow can be viewed, instead of time-averaged view (as with the prior art MHC method in which the thermal diffusion method is used to determine blood flow velocity).

In an alternative exemplary embodiment of the present invention, said means for determining blood flow velocity comprises optical Doppler tomography measurement means wherein said blood flow velocity is determined according to a change of frequency of radiation reflected from said portion of said live subject.

Beneficially, the means for measuring the concentration of haemoglobin and oxygenated haemoglobin comprises optical means, which may comprise one or more of spectral reflectance spectroscopy means, Raman spectroscopy means, photo-acoustic spectroscopy means, thermal emission spectroscopy or optical coherence tomography means.

Thus, because blood flow rate is determined by optical means, it is dependent on a single parameter, namely the detection signal, such that the blood flow rate and therefore the glucose concentrate, can be measured more quickly and accurately.

Preferably, said irradiating means comprises a laser cavity.

Preferably, said measuring beam comprises a laser beam.

Preferably, said detector means comprises a laser cavity and/or a photodetector.

Preferably, said portion of said live subject is placed in the focal plane of a laser beam.

Preferably, said tissue sample is a finger tip.

Preferably, said measuring beam radiation has a wavelength substantially in the range 470-950 nm.

These and other aspects of the present invention will be apparent from, and elucidated with reference to the embodiment described herein.

An embodiment of the invention will now be described by way of example only and with reference to the accompanying drawings, in which:

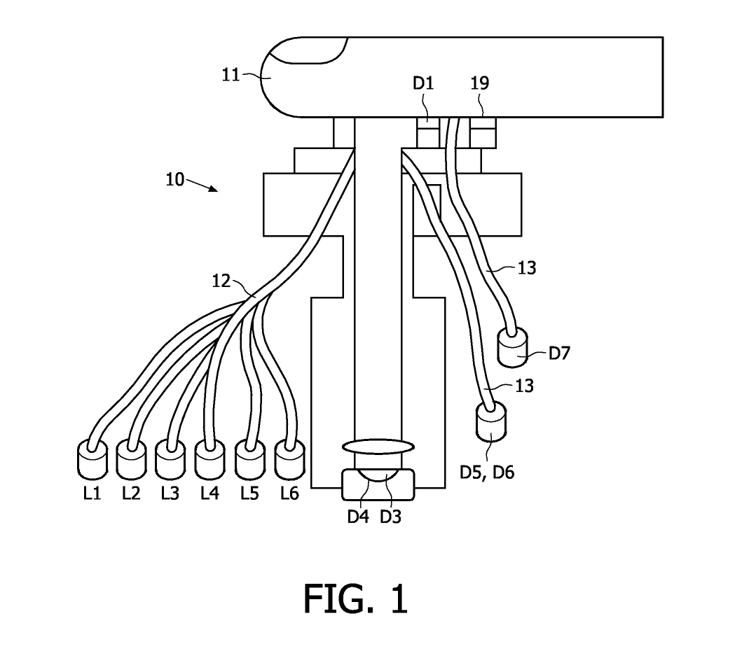

FIG. 1 is a schematic representation of the apparatus for determining the blood glucose concentration in accordance with the present invention; and,

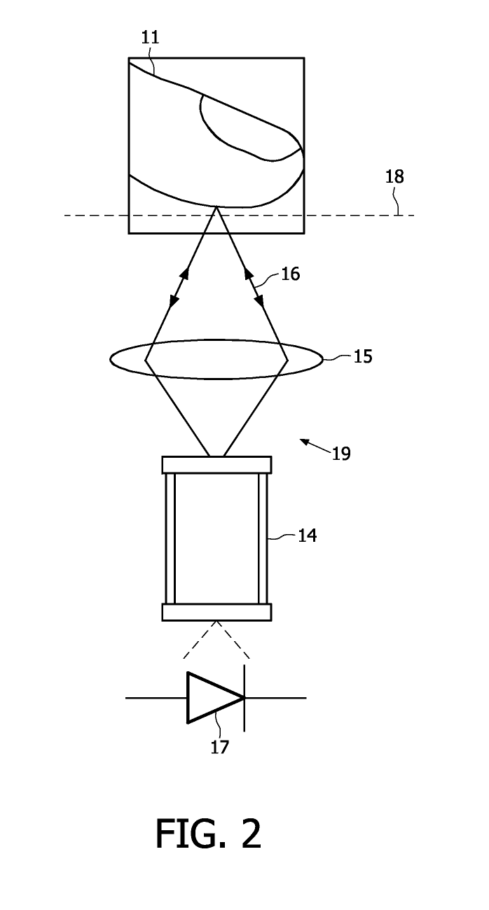

FIG. 2 is a schematic representation of the self-mixing interferometric apparatus.

Referring to FIG. 1 of the drawings, there is illustrated schematically a system 10 for performing non-invasive measurement of blood glucose concentration in a live subject. Using thermistors D1 and D4, and thermopile D3, the temperature of the finger tip surface 11 can be measured in order to determine the heat generated. The light emitting diodes (LED's) L1-L6 and photodiodes D5-D7 are used to measure the Hb and HbO₂ concentrations using spectral reflectance spectroscopy, however, Raman spectroscopy, photo-acoustic spectroscopy, thermal emission spectroscopy and optical coherence tomography can also be used.

The light generated by the LED's L1-L6 is communicated to the surface of the finger 11 using a set of optical fibres 12 and the light reflected from the surface of the finger is returned to the photodiodes D5-D7 using a second set of optical fibres 13. The wavelength of the light used in the determination of Hb and HbO₂ concentration is typically in the range 470 nm-950 nm—a range which includes the visible and infra red regions of the electromagnetic spectrum.

The blood flow rate can, for example, be determined directly by means of self-mixing interferometry or optical Doppler tomography.

Koelink et al. (“Signal Processing for a Laser-Doppler Blood Perfusion Meter”, Signal Processing, 38, pp 239-252 (1994)) have demonstrated the application of self-mixing interferometry for the direct measurement of blood velocity. Zhao et al. (“Phase-Resolved Optical Coherence Tomography and Optical Doppler Tomography for Imaging Blood Flow in Human Skin with Fast Scanning Speed and High Velocity Sensitivity”, Opt. Lett., 25(2), pp 114-116 (2000)) have demonstrated the use of Doppler tomography to directly determine the blood flow rate.

Self-mixing interferometry and optical Doppler tomography both involve the direct optical determination of blood flow rate. With regards to the former, the optical analysis relies on the interference of the radiation incident upon the tissue sample with that reflected therefrom. Optical Doppler tomography however, exploits the frequency change suffered by radiation reflected off a moving object.

In the embodiment illustrated in FIG. 1 of the drawings, the blood flow rate is determined using a self-mixing interferometry unit 19, which is shown in more detail in FIG. 2. The unit comprises a laser cavity 14, a lens system 15 to focus the laser beam 16 onto the tissue sample, i.e. the finger tip surface 11, and a photodetector 17. The laser beam 16 is focussed onto a focal plane which contains a surface 18 to which the finger 11 is applied. The surface 18 ensures that the surface of the finger 11 is suitably positioned at the focal plane of the lens system 15.

The beam emanating from the laser cavity 14 reflects off the surface of the finger 11 and is entrained back into the laser cavity 14 by the lens system 15. The interference of the laser beam 16 with the reflected beam within the laser cavity 14, sets up power fluctuations in the laser output, which is measured using the photodetector 17. The technique bears the name self-mixing interferometry due to the fact that the light reflected back into the laser cavity 14 interferes with the light resonating within the cavity.

If no blood flows in the finger 11 and the finger 11 is not moved, then everything is static, and the resulting signal from the photodetector 17 will be a constant in time (zero if DC filtered). If the finger 11 moves, or the amount of blood changes in the finger 11, then the amount of reflected light is changed and this will create fluctuations in the laser 14. The measured fluctuations will mirror these movements, and so the heartbeat will be an implicit part of the signal.

The signal on the photodetector 17 can also be understood on the basis of the speckle pattern when the blood flows. If no blood flows in the finger 11, then the speckle pattern will remain constant and the signal will be constant. When the blood flows, the speckle pattern will change in proportion to the blood flow velocity. The larger the velocity of the blood, the faster it changes the speckle pattern and the faster the signal on the photodetector 17 will oscillate (the oscillation period being typically between 0.1 ms and 2 ms). Thus, if the pattern is Fourier transformed, then as the signal oscillation rate increases, so will the number of high frequency components in the transform.

By measuring the signal from the photodetector 17, the heartbeat and blood velocity can be measured simultaneously. This allows for a real time-varying rate of blood flow to be viewed, instead of a time-averaged view, as with the thermal diffusion method. More importantly however, the direct optical determination of blood velocity provides a more accurate determination of the blood velocity than the known thermal diffusion method associated with the MHC method and also provides for a more rapid measurement.

Thus, having determined the heat generated, the Hb and HbO₂ concentrations and blood velocity, the blood glucose concentration can be determined. It should be noted however, that whilst the measurement of blood velocity has been described here using a self-mixing interferometric technique, optical Doppler tomography could equally be employed. This technique involves the illumination of a tissue sample and collecting the backscattered radiation at a detector. The reflection of waves off a moving object is known to cause a frequency shift (the typical example being the change in the tone of a police car siren as the car approaches and then moves away), from which the speed of the moving object can be determined. Thus, due to the interaction of the radiation with the moving red blood cells within the radiated tissue sample and maybe the pulsating surface of the tissue sample, some regions of the radiation will suffer a frequency shift causing the intensity of the backscattered light to fluctuate. This fluctuation can then be used to determine blood flow velocity.

The use of self-mixing interferometry to measure blood flow rate, as opposed to the well-known thermal diffusion method, and other spectroscopic methods to measure Hb and HbO₂ concentrations, will speed up the determination of blood glucose levels. Additionally, because the measurement of blood flow rate only depends on the one parameter (namely, the self-mixing interferometric signal), the system will also improve the accuracy of the MHC method.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. A system (10) for non-invasive measurement of glucose concentration in a live subject (11), said system (10) comprising temperature sensing means (D1, D3, D4) for measuring body heat in respect of said subject (11), means (L1-L6, D5-D7) for measuring the concentration of haemoglobin and oxygenated haemoglobin in the blood of said live subject (11), irradiating means (14) for generating a measuring beam (16) and irradiating therewith a portion of said live subject (11), detector means (14, 17) for collecting measuring beam (16) radiation reflected by said live subject (11), means for determining from said reflected measuring beam radiation blood flow velocity in respect of said live subject (11), and means for determining glucose concentration in said live subject (11) as a function of said body heat, said haemoglobin and oxygenated haemoglobin concentrations, and said blood flow velocity.

2. A system as claimed in claim 1, wherein said means for determining blood flow velocity comprises self-mixing interferometry.

3. A system as claimed in claim 1, wherein said means for determining blood flow velocity comprises optical Doppler tomography

4. A system as claimed in claim 1, wherein the heartbeat and blood velocity are measured substantially simultaneously in said live subject (11).

5. A system as claimed in claim 1, wherein means for measuring the concentration of haemoglobin and oxygenated haemoglobin comprises optical means.

6. A system as claimed in claim 5, wherein said optical means comprises one or more of spectral reflectance spectroscopy means, Raman spectroscopy means, photo-acoustic spectroscopy means, thermal emission spectroscopy or optical coherence topography means.

7. A system as claimed in claim 1, wherein said irradiating means comprises a laser cavity (14).

8. A system as claimed in claim 1, wherein said measuring beam comprises a laser beam (16).

9. A system as claimed in claim 1, said detector means comprises a laser cavity (14) and/or a photodetector (17).

10. A system as claimed in claim 1, wherein said portion of said live subject (11) is placed in the focal plane (18) of a laser beam (16).

11. A system as claimed in claim 1, wherein said tissue sample is a finger tip (11).

12. A system as claimed in claim 1, wherein said measuring beam radiation (16) has a wavelength substantially in the range 470-950 nm.
